(19) United States
(12) Patent Application Publication
(10) Pub. No.: US 2026/0251917 A1
(43) Pub. Date: Aug. 27, 2026

(54) VITRECTOMY LENS AND LENS CARRIER

(71) Applicant: Katena Products, Inc., Parsippany, NJ (US)

(72) Inventor: Gregory Heacock, Maple Valley, WA (US)

(21) Appl. No.: 19/065,921

(22) Filed: Feb. 27, 2025

(51) Int. Cl. G02C 7/04

(52) U.S. Cl. CPC G02C 7/047; G02C 7/049

(57) ABSTRACT

The present application concerns a novel ophthalmic device comprising a contact lens and lens carrier, wherein the lens carrier is configured to surround and hold at least a portion of the lens within an interior portion of the lens carrier. One end of the lens carrier is configured to facilitate movement of the device and another end is configured to facilitate contact between the vitrectomy lens and the eye. The lens carrier retains the vitrectomy lens within the lens carrier when the device is manipulated.

### CROSS REFERENCE TO RELATED APPLICATIONS

[Not Applicable]

### BACKGROUND

Ophthalmic lenses are used by ophthalmologists and optometrists for diagnoses and treatment of the eye. A common procedure performed is the examination and treatment of the interior of a patient's eye. These are important procedures since variations in the appearance of the interior of a patient's eye can give the treating professional important information on the health of a patient or the proliferation of a number of ocular diseases.

In a vitrectomy procedure, clinicians use a vitrectomy lens, which is placed in contact with the patient's cornea, to view, treat, and examine the patient's eye, e.g., the retina and vitreous. For example, the vitreous is often removed to gain access to the retina and thereafter replaced with another solution. Oftentimes, in such procedures, the clinician will need to manipulate the lens and ultimately the patient's eye to examine various portions of the eye, e.g., posterior or anterior regions.

In conventional procedures, a bottom or distal portion of the vitrectomy lens is seated in an open silicone (or sometimes stainless steel) lens “ring.” The vitrectomy lens may be generally shaped as two stacked but integral cylinders of different diameters. In such a case, the bottom cylinder, which includes the eye-contacting portion of the lens, is shorter and has a smaller diameter than the main body of the lens. The smaller bottom portion of the lens fits within the ring while a bottom surface of the main lens body sits on top of a top ledge of the ring. The ring allows the lens to contact the eye but also provides a base that allows the clinician to stabilize the lens on the eye while also moving the lens. An example of such conventional lens and ring systems include Ocular Instruments Landers Vitrectomy lens ring system.

However, conventional lenses prove difficult and inconvenient for the clinician and often create complications for the patient. Because it is simply seated on top of the ring, the lens can dislodge from the ring complicating the procedure. Moreover, movement of the lens and ring on the eye is not always practical. Clinicians aim to avoid touching the lens so that optical clarity is not compromised but movement is otherwise difficult. Work-arounds have not been ideal. For example, clinicians often have to anchor and move the device by suturing the ring to the eye to provide the needed stability during the procedure. Moreover, there is no practical way to hold the eye in place relative to the lens and ring assembly (e.g., without suturing the device in place) so that the eye can be moved with the lens in place and remote portions of the eye can be examined and treated.

A need therefore exists for an ophthalmic device including a vitrectomy lens that can provides needed stability on the patient eye while allowing the clinician to manipulate the eye and the lens without compromising the clarity of the lens and at the same time avoiding dislodging the lens from its ring.

### SUMMARY

The disadvantages of the prior art have been overcome. The present invention is an ophthalmic device that includes a contact lens and novel lens carrier, which allows the clinician to manipulate the eye and the lens without affecting optical clarity while at the same time offering a better patient experience.

According to an embodiment, an ophthalmic device comprises a contact lens and a lens carrier configured to hold the contact lens. The contact lens includes a lens upper surface and a lens lower surface, wherein the lens lower surface comprises a first curvature configured to contact an eye. The lens carrier has a first end and a second end, and further comprises a main body portion comprising a sidewall configured to extend circumferentially about at least a portion of the contact lens, and a hollow interior region within the sidewall. The main body portion is configured to receive at least a portion of the lens within the hollow interior region. In some embodiments, the lens carrier includes a tab proximate to the first end of the lens carrier and extending outwardly from a center axis of the main body portion and/or a skirt proximate to and extending from the second end of the lens carrier, wherein the skirt is configured to contact the eye.

In preferred embodiments, the contact lens is a vitrectomy lens. In embodiments, the lens upper surface includes a planar curvature.

In some embodiments, the tab extends in a radial direction from the center axis of the main body portion. The length of the tab is at least about 2 mm in some embodiments. In embodiments, the tab is configured to facilitate movement of the eye.

In embodiments, the lens carrier may further comprise a lip extending above the hollow interior region at the first end, the lip extending inwardly towards the center axis of the main body portion. In embodiments, the lip forms an aperture to view the eye through the contact lens.

In embodiments, the lens carrier is formed of a flexible material and is configured to flexibly fit the lens. In preferred embodiments, the lens carrier is a molded lens carrier integrally formed as a single piece. In some embodiments, wherein an inner diameter of the lens carrier is less than an outer diameter of the lens. These features simplify manufacturing of the carrier and installation and fit of the lens within the lens carrier.

In some embodiments, the lens carrier further comprises a recess or rib on a sidewall of the main body portion and the lens further comprises a corresponding rib or recess on a lateral surface of the lens that engages the recess or rib of the lens carrier.

According to an embodiment, the ophthalmic device comprises a vitrectomy lens that includes a lens upper surface and a lens lower surface, wherein the lens lower surface comprises a curvature configured to contact an eye, and a lens carrier. The lens carrier comprises a main body portion including a sidewall and a hollow interior region, wherein the lens carrier receives at least a portion of the vitrectomy lens. The lens carrier comprises a lip at the proximal end of the lens carrier, the lip extending inwardly towards a center axis of the ophthalmic device.

In some embodiments, the lens carrier further comprises a tab extending away from the center axis of the ophthalmic device. In some embodiments, the tab extends in a radial direction from the center axis of the main body portion. The length of the tab is at least about 2 mm in some embodiments. In embodiments, the tab is configured to facilitate movement of the eye.

In some embodiments, the vitrectomy lens carrier further comprises a skirt portion configured to contact the eye.

In some embodiments, a length of the lip is between about 1 and 2 mm. In some embodiments, the lip forms an aperture to view the eye through the vitrectomy lens.

In some embodiments, the lens carrier comprises a flexible material and is flexible. In preferred embodiments, the lens carrier is a molded lens carrier integrally formed as a single piece.

In some embodiments, an inner diameter of the lens carrier is less than an outer diameter of the lens.

According to an embodiment, an ophthalmic device comprises a lens carrier having a first end and a second end, wherein the lens carrier is configured to surround and hold at least a portion of a vitrectomy lens within an interior portion of the lens carrier. The first end of the lens carrier is configured to facilitate movement of the device and the second end of the lens carrier is configured to facilitate contact between the vitrectomy lens and the eye. The lens carrier being further configured to retain the vitrectomy lens within the lens carrier when at least a portion of the ophthalmic device is manipulated.

In some embodiments, the lens carrier comprises a tab extending outwardly from a center axis of the ophthalmic device.

In some embodiments, the lens carrier comprises a main body portion and a lip, wherein the main body portion is configured to encompass a proximal end of the vitrectomy lens, and the lip is located proximally from the main body portion, the lip being positioned proximally from the proximal end of the vitrectomy lens when the lens is within the carrier.

In some embodiments, the lens carrier is flexible and comprises silicone and wherein an inner diameter of the lens carrier is less than an outer diameter of the lens.

The novel ophthalmic device including both the lens and novel lens carrier provides significant advantages over the prior art, making the system easier for the clinician to use and less traumatic for the patient. The novel configuration of the lens carrier keeps the lens in place within its holder while allowing the clinician to manipulate the lens and the eye without compromising the clarity of the lens or using complex suturing that will further inconvenience or traumatize the patient.

### BRIEF DESCRIPTION OF SEVERAL VIEWS OF THE DRAWINGS

FIG. 1 illustrates a top perspective view of an ophthalmic device, according to embodiments.

FIG. 2 illustrates an exploded view from a top perspective of the ophthalmic device of FIG. 1, according to embodiments.

FIG. 3 illustrates a cross-sectional view of an ophthalmic device, according to embodiments.

FIG. 4 illustrates an embodiment of the ophthalmic device placed on an eye (cross-sectional view).

FIGS. 5A, 5B, and 5C illustrate embodiments of an ophthalmic device, according to embodiments, placed on an eye (cross-sectional view) wherein the lens is a planar lens, magnifier lens, and biconvex lens, respectively.

FIG. 6 illustrates an ophthalmic device, along the line 3 -3 shown in FIG. 1, according to embodiments.

FIG. 7 illustrates an ophthalmic device, similar to that of FIG., with dimension markings added to the ophthalmic device, according to embodiments.

FIG. 8 illustrates another embodiment of the ophthalmic device (cross-sectional view), according to embodiments.

The foregoing summary, as well as the following detailed description of certain techniques of the present application, will be better understood when read in conjunction with the appended drawings. For the purposes of illustration, certain techniques are shown in the drawings. It should be understood, however, that the claims are not limited to the arrangements and instrumentality shown in the attached drawings. Furthermore, the appearance shown in the drawings is one of many ornamental appearances that can be employed to achieve the stated functions of the system.

### DETAILED DESCRIPTION

The invention relates to a novel ophthalmic device comprising a contact lens including a vitrectomy lens and a novel lens carrier. The lens includes a clear aperture that allows a clinician to examine and/or treat the eye and includes an upper and lower surface. The lower or distal surface of the lens has a curvature configured to contact an eye (e.g., the cornea). The lens carrier comprises a main body (or main body portion) comprising a sidewall and hollow interior region. The lens fits within the hollow interior region of the main body portion, allowing the main body portion to extend circumferentially around the lens and the lens to appropriately contact the patient's eye.

The lens carrier is configured to surround and hold at least a portion of the vitrectomy lens within an interior portion of the lens carrier and may be configured to facilitate movement of the ophthalmic device. The lens carrier may retain the vitrectomy lens within the lens carrier when at least a portion of the lens or lens carrier is manipulated.

In some embodiments, the lens carrier further comprises a tab (or tab portion of the carrier) proximate to and extending outwardly from a center axis of the main body portion or ophthalmic device (e.g., outwardly from the sidewall). The tab portion may be configured to facilitate movement of the eye.

In some embodiments, the lens carrier further comprises a lip (or lip portion of the carrier), which may be located proximally from the hollow interior region and extend inwardly towards a center axis (e.g., inwardly in the direction of the hollow region).

The tab and lip can be located on the proximal side of the device nearer to the clinician.

In some embodiments, the lens carrier further comprises a skirt (or skirt portion of the carrier) located on the other end of the lens carrier (the distal end). The skirt may facilitate movement of the device as well as contact of the lens with the eye.

In some embodiments, the novel ophthalmic device comprises a vitrectomy lens and a lens carrier for the vitrectomy lens. The lens carrier is configured to surround and hold at least a portion of the lens within the carrier. The end of the lens carrier closest to the clinician (proximate end) is configured to facilitate movement of the device by the clinician. While the other end of the lens carrier (distal end) is configured to facilitate contact between the lens and the eye. In embodiments, the distal end of the lens carrier can attach to (e.g., suction) the eye such that the ophthalmic device maintains a static positional relationship with respect to the eye. The novel lens carrier is able to securely retain the lens within the carrier when the clinician manipulates the lens or lens carrier.

The novel ophthalmic devices allow the clinician to safely and effectively manipulate the lens or eye without compromising lens clarity or inconveniencing the patient with suturing and other unnecessary procedures.

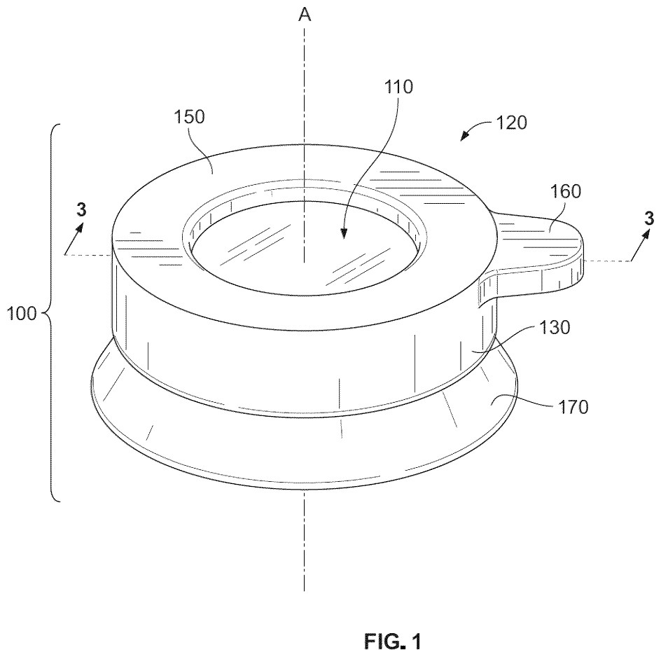

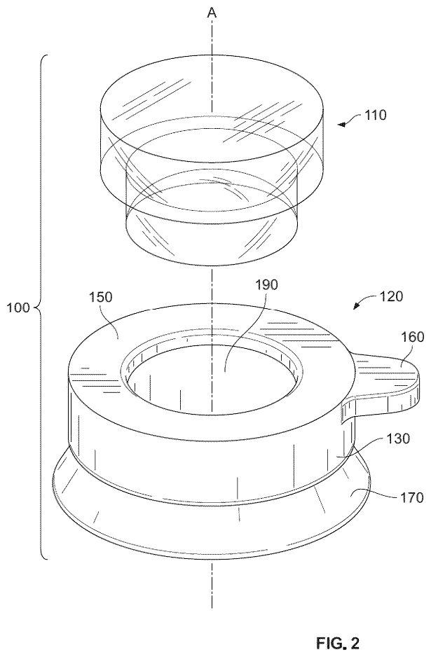

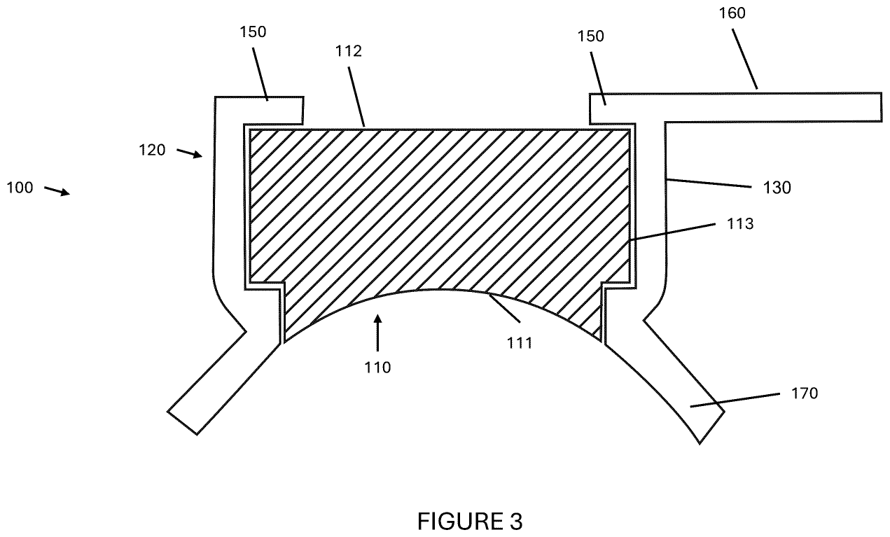

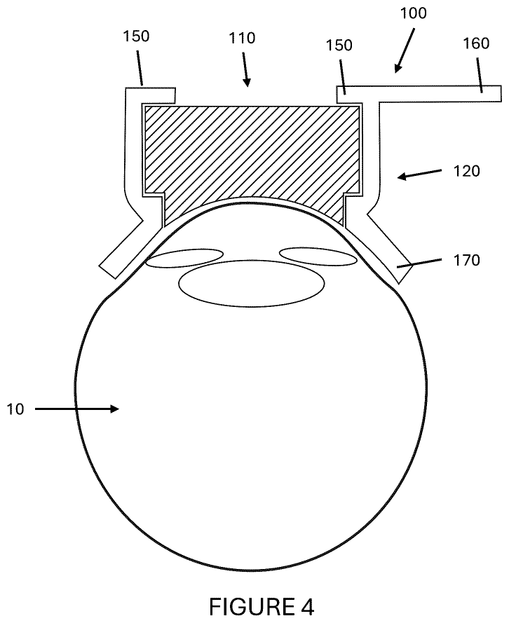

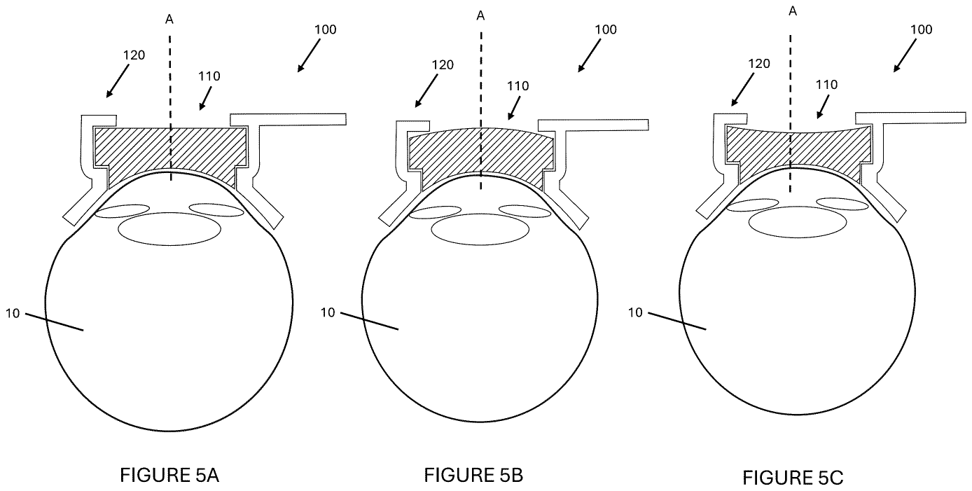

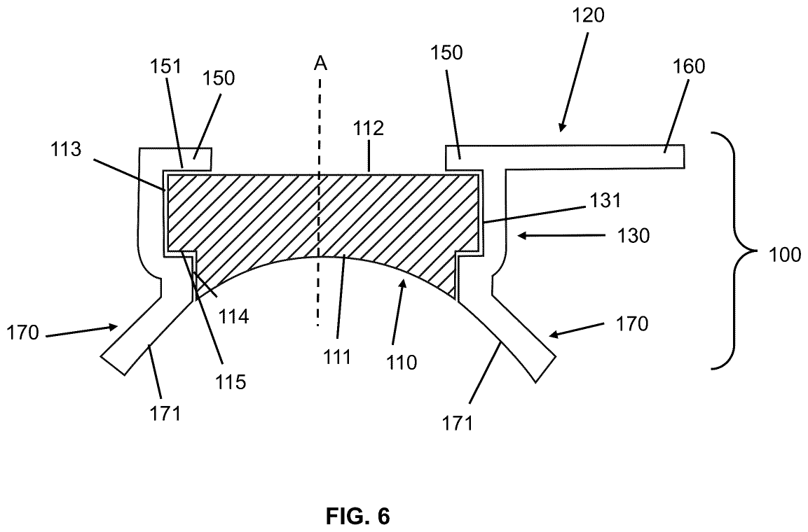

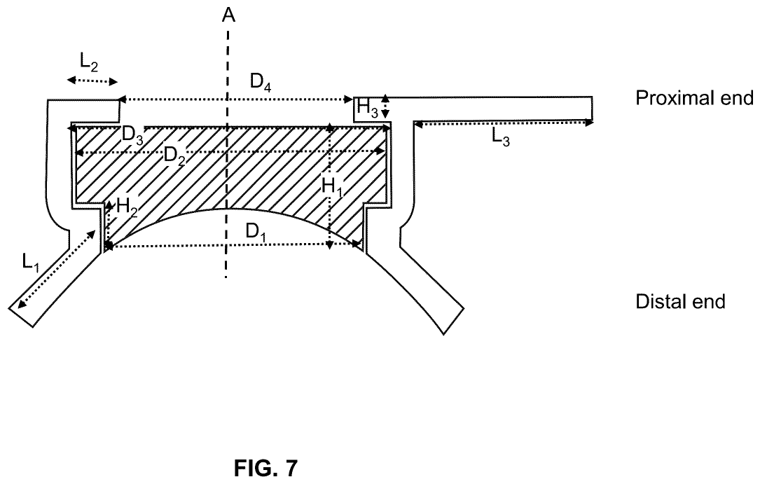

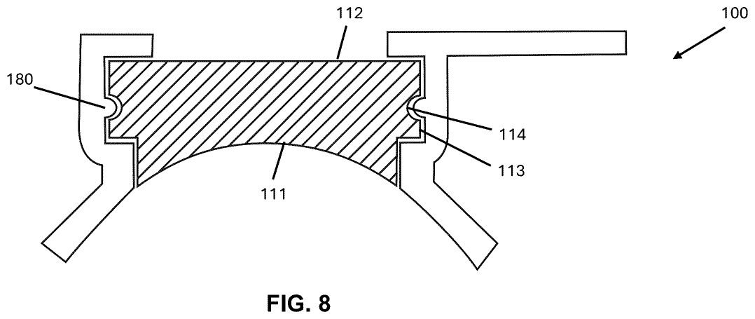

FIG. 1 illustrates a top perspective view of an ophthalmic device 100, according to embodiments. FIG. 2 illustrates an exploded view from a top perspective of the ophthalmic device 100 of FIG. 1, according to embodiments. FIG. 3 illustrates a cross-sectional view of an ophthalmic device 100, according to embodiments. FIG. 4 illustrates an embodiment of the ophthalmic device 100 placed on an eye 10 (cross-sectional view). FIGS. 5A, 5B, and 5C illustrate embodiments of the ophthalmic device 100 placed on an eye 10 (cross-sectional view) wherein the lenses 110 are a planar lens, magnifier lens, and biconvex lens, respectively. FIG. 6 illustrates an ophthalmic device 100, along the line 3-3 shown in FIG. 1, with certain features identified. FIG. 7 is similar to FIG. 6 with dimension markings added to the ophthalmic device, according to embodiments. FIG. 8 illustrates another embodiment of the ophthalmic device (cross-sectional view).

In one embodiment, an ophthalmic device includes a lens and a lens carrier. The lens and lens carrier can be separately packaged and provided together or separately for the clinician.

The lens is formed of an optically transparent material and is positioned in close proximity to the patient's eye. The lens 110 generally has a lens lower surface 111, a lens upper surface 112, as well as one or more lens lateral surfaces (e.g., 113 and 114). The lens lower surface is a distal surface positioned facing the patient's eye adjacent to the cornea, and the lens upper surface is a proximal surface that faces the observing clinician. In some embodiments, the lens may further include lateral lens surface or surfaces 113 or 114, at least a portion of which is recessed inwardly, for example as shown in FIGS. 6 and 8. The recessed lens lateral surface may be located in any portion of the lens including, for example, a distal region of the lens (e.g., 114 in FIG. 6) or proximal or middle region of the lens (e.g., 114 in FIG. 8). In some embodiments, the ophthalmic device may be used for an ophthalmic procedure, such as vitrectomy surgery, and the lens is a vitrectomy lens.

The lens and/or the lens carrier can each be separately and integrally formed including each as a single piece lens or a single piece lens carrier and each can be injection or compression molded to improve ease of manufacture.

As shown for example in FIGS. 4, 5A, 5B, and 5C, a molded ophthalmic contact lens 110 seated within lens carrier 120 is positioned in contact with the patient's eye 10. The lens is formed of an optically transparent material and has a contact surface (e.g., 111 as shown in FIG. 8) that touches the cornea of a patient and second lens surface or opposing surface (e.g., 112 in FIG. 8) that faces the observing clinician. The lens may be substantially hard and formed of an optically transparent material, enabling a clinician to view certain parts of a patient's eye (e.g., the retina). For example, the optically transparent material may be any moldable material. For example, it can be a plastic material or other suitable transparent material. In one embodiment, the material is or includes an optical grade acrylic resin such as polymethylmethacrylate (PMMA), styrene, polycarbonate or others well known in the art. The material (e.g. acrylic resin such as PMMA) can have a specific gravity of 1.1-1.9, or preferably 1.1-1.2, or preferably less than 1.19. The material can also have an index of refraction of 1.4-1.55, or preferably 1.49.

In some embodiments, the lens is integrally formed of a molded, optical grade acrylic resin such as polymethylmethacrylate, having a specific gravity of between about 1.1-1.8. In another embodiment, the specific gravity of the material (e.g., acrylic resin) is between about 1.15-1.19 and the index of refraction is between about 1.2 and 2.0. The lens may be injection molded and/or compression molded.

Optionally, the lens may be formed of or include glass. In such a case, the lens may be ground at least partially or completely (e.g., the lens lower surface and/or the lens upper surface may be ground).

The lens can be of any suitable size or shape. For example, in one embodiment, the lens can be cylindrical with only one outer diameter. In another embodiment, the lens can be shaped like some conventional vitrectomy lenses having at least two outer diameters, one defining a narrower cylinder in the lower region of the lens and one defining a wider cylinder above the narrower cylinder.

In other embodiments, the lens such as a vitrectomy lens comprises circumferential recess or recesses (e.g., 114) around all or a portion of the lens lateral surface. Such recesses engage with a corresponding rib or ribs (e.g., 180) on the inner surface of the lens carrier, for example on the sidewall 131. In alternate embodiments, the lens includes a circumferential rib or ribs around all or a portion of the lens lateral surface that engage or mate with a corresponding recess or recesses on the inner surface of the lens carrier. These recesses and ribs in the lens, e.g. vitrectomy lens, and corresponding ribs and recesses in the lens carrier facilitate the ability of the lens 110 to stay mounted within its lens carrier.

The lens has an axis which, during surgery, can align with the optical axis of the eye, both of which are referred to herein as axis A. As will be further discussed, the hollow interior region of the lens carrier can also have an axis that aligns with the optical axis of the eye, and this axis is also referred to as axis A, herein. However, it is understood that some or all of these axes need not align, either during or not during surgery (e.g., vitrectomy surgery). These axes are all referred to as axis A herein and correspond to a particular embodiment or use case, and are commonly referenced herein for the purpose of clarity.

The lens can have a vertical thickness or thicknesses along the lens lateral surface (e.g., 113 and/or 114) that is at least about 4 mm for vitrectomy lenses, such as between about 3 to 9 mm in preferred embodiments. As mentioned, the lens may have two or more diameters. The lens can have a diameter (e.g., in the proximal region of the lens) of at least about 8 to 12 mm, such as between about 6 to 14 mm, in preferred embodiments. The lens can have a diameter (e.g., in a distal region of the lens) of at least about 6 to 10 mm, such as between about 4 to 12 mm, in preferred embodiments.

The lens lower or distal surface comprises a curvature configured to contact an eye and is an eye-contacting portion. It may be shaped to contact the cornea of a patient's eye (e.g., contacting a fluid on the surface of the eye). In this sense, the lens is a contact lens. The lens lower surface may have a curvature that substantially matches the curvature of a cornea at least in the region of the cornea on which the lens is located.

The lens lower surface may traverse a distance D₁ (shown in FIG. 7) of between 4 and 12 mm, such as about 6 to 10 mm, in preferred embodiments. As will be discussed, D₁ is the diameter of the lens at a distal end. The lens lower surface may have a radius curvature of between about 6.5 to 9 mm, and preferably about 7.8 mm, in embodiments.

The lens upper or proximal surface, through which the clinician views the eye, may have one or more of a variety of contours or curvatures such as planar (see, e.g., FIG. 5A), magnifying (see, e.g., FIG. 5B), or biconcave (see, e.g., FIG. 5C) and thus may be a planar lens, magnifier lens, or biconcave lens. The contour or curvature of the lens upper surface may be different than the contour or curvature of the lens lower surface. The lens upper surface is located on the proximal end of the lens and faces the clinician when the ophthalmic device is being used. . In one embodiment of a lens 110 for a vitrectomy procedure, the lens 110 is a planar lens with an upper surface 112 that has a planar or zero curvature.

The combination of the lens lower surface, lens upper surface, index of refraction of the lens, and the index of refraction of the media abutting the lens lower surface, together, determine the optical power of the lens. According to some embodiments, the lens has an optical power of a value between +10 and −43 diopters. In these cases, the optical power of the lens (when in contact with the eye) may be selected to substantially offset the optical power of a cornea 10 (e.g., a typical human cornea). In such a case, the lens upper surface may be substantially planar.

In embodiments, the lens lateral surface or surfaces (e.g., 113 and 114) extend from the lens lower surface to the lens upper surface. In the embodiment of FIG. 3, the lens lateral surface 113 is the surface(s) that extends around the outer side of the lens 110. In the embodiment depicted in FIG. 6, the outer surface of the lens 110 includes lens lateral surfaces 113 and 114 as well as ledge surface 115. As discussed further below, the ledge allows the lens to be seated within the lens carrier.

As discussed the lens and lens carrier may have several diameters including, for example, as shown in FIG. 7. D₁ is the diameter at a distal or lower end of the lens including to a lens lateral surface. D₂ is the diameter at a proximal or upper region of the lens including to a lens lateral surface. D₃ is the diameter or distance of the interior region of the lens carrier at its proximal end. D₄ is the diameter or distance of an aperture in the ophthalmic device. In some embodiments, D₁ may be between 4 and 12 mm, such as about 6 to 10 mm or at least about 6 mm. D₂ may be between 6 and 14 mm, such as about 8 to 12 mm or at least about 8 mm. Other dimensions are discussed herein.

Referring to FIG. 7, the height H₁ of the lens (and the lens lateral surfaces) may be between 2 and 8 mm or 3 and 9 mm, such as about 4 mm, about 5 mm, or about 4 to 5 mm, in preferred embodiments. The height H₂ of the lens lateral surface 114 (in a stepped lens embodiment) may be between 1 and 2 mm in preferred embodiments.

The lens ledge surface segment (e.g., 115) can form a step between the upper region and lower region of the lens. The lens lateral surface segment may be substantially horizontal or may be at an angle. D₁ and D₂ may be equal, and in such a case, there is no lens lateral surface 113.

In other embodiments, the lens lateral surface may include a recess or recessed portion such as the recess 114 shown in FIG. 8. The recess may receive a corresponding main body sidewall rib (e.g., 180). There may be a plurality of recesses and corresponding ribs. Alternatively, or in addition, the lens lateral surface may have rib(s) that are received by corresponding recesses in a sidewall of the lens carrier. Thus, in some embodiments, the ophthalmic device includes a lens carrier with a recess or rib on a sidewall of the main body portion of the lens carrier and the lens further comprises a corresponding rib (in the case of a lens carrier recess) or recess (in the case of a lens carrier rib) on a lateral surface of the lens that engages or mates with the corresponding recess or rib of the lens carrier.

As discussed, the ophthalmic device comprises a lens carrier. The lens carrier is configured hold at least a portion of the contact lens (such as a vitrectomy lens) or hold and surround at least a portion of the lens (such as a vitrectomy lens) within an interior portion of the lens carrier.

The lens carrier holds or carries the lens, for example, by receiving the lens in a hollow interior region (e.g., 190 as shown for example in FIG. 2). In some embodiments, the entirety of the lens is received within the lens carrier.

The lens carrier has a first end and a second end as well as a main body or main body portion 130, for example as shown in FIG. 1. In some embodiments, the first or proximal end of the lens carrier is configured to facilitate movement of the ophthalmic device and the second or distal end is configured to facilitate contact with the eye. The lens carrier is configured to retain the lens (such as a vitrectomy lens) within the lens carrier when at least a portion of the lens or lens carrier is manipulated.

The lens carrier comprises a hollow interior region (e.g., 190), wherein the lens carrier receives at least a portion of the lens (e.g., a vitrectomy lens) within the hollow interior region. The main body portion (e.g., 130) of the lens carrier may hold or encompass all or a large portion of the lens such that the lens does not dislodge from the ophthalmic device. In some embodiments, the lens carrier has a first and second end and is configured to surround and hold at least a portion of a vitrectomy lens within an interior portion of the lens carrier. In some embodiments, the main body portion is configured to encompass the proximal end of a lens (e.g., vitrectomy lens) such as shown in FIG. 3.

In some embodiments, the lens carrier 120 may further include a lip 150, a tab 160, and/or a skirt 170, either alone or in combination, in addition to the main body portion. For example, in some combinations, the lens carrier comprises a lip, tab, and skirt; a lip and tab; a lip and skirt; a and a tab and skirt. These components may be integrally formed in a single body of the lens carrier (such that a molded lens carrier is integrally formed as a single piece), or may be separate but joined components. It is understood that the lip, tab, and skirt may be alternatively referred to as the lip portion, tab portion, and skirt portion of the lens carrier.

The main body portion includes a sidewall (e.g., 131). The sidewall is configured to extend circumferentially about at least a portion of the contact lens including the upper or proximal end of the lens. There is a hollow interior region within the sidewall that allows the main body portion to receive at least a portion, and preferably substantially all, of the lens including the proximal or upper portion of the lens. The lens fits within the hollow interior region, holding the lens in place.

In some embodiments, the distance or diameter between main body sidewall on either side of the lens carrier (D₃ shown, e.g., in FIG. 7) may be between about 6 and 14 mm, such as about 8 and 12 mm at its widest. In some embodiments, the inner surface of the main body sidewall can have different diameters. For example, the diameter of the hollow interior region (e.g., 190) between the inner surfaces of the main body sidewall (e.g., 131) may vary depending on the diameters of the associated lens. For example, as depicted in FIG. 7, in some regions the diameter may be larger (e.g., the upper or proximal region of the main body portion (D₃)) or smaller (e.g., the lower or distal region in FIG. 7). As discussed herein, in some embodiments, the diameter of the lens carrier can also be configured such that it is slightly smaller in diameter than the lens (e.g., by about 0.5 mm or less) to enable a snug fit between the carrier and the lens.

At the proximal end of the ophthalmic device, the main body portion forms an aperture through which light passes into and back out from the lens. The aperture may be substantially circular. The aperture may be sufficiently large such that, if a procedure is being performed, the clinician will be able to adequately see through a sufficient portion of the lens to view relevant features in the patient's eye 10. Referring to FIG. 7, the aperture could have a diameter of D₃ (or as discussed herein, D₄).

As discussed, the lens carrier 120 may include sidewall rib(s) 180 in the main body portion 130 that mate with corresponding lens lateral surface recess(es) 114, as shown in FIG. 8. Alternatively, or in addition, the sidewall may have recess(es) and the lens lateral surface may have corresponding rib(s) that function in a similar manner. These mating features allow the lens to be held in place within the lens carrier. As shown in FIG. 8, the rib/recess features can be implemented as an alternative to the stepped arrangement of the lens for example as shown in FIG. 6. The rib/recess features can be provided in addition to such a stepped arrangement.

Other types of complementary features between the lens and the main body may be implemented to facilitate the lens carrier to hold the lens. For example, the inner surface of the sidewall (or a portion thereof) may have a rough surface which contacts a corresponding rough surface of the lens lateral surface. As another example, only one of the inner surface of the sidewall or the lateral surface may have a rough or gripping feature to facilitate the holding of the lens by the lens carrier.

In some embodiments, the lens carrier may further comprise one or more lips. The lip may be located proximally from the main body portion and positioned proximally from the proximal end of the lens (e.g., vitrectomy lens) when the lens is within the carrier. The lip may be positioned above (proximal to) the hollow interior region of the main body portion and extend inwardly towards a center axis of the ophthalmic device.

In some embodiments, the lip may assist in securement of the lens within the lens carrier. As shown, for example in FIG. 6, the lip 150 may extend above the hollow interior region of the main body portion 130 at one end of the lens carrier 120 (e.g., the proximal end). The lip 150 extends inwardly towards a center axis A of the main body portion 130. In some embodiments such as shown in FIG. 1, the lip 150 extends inwardly toward the center axis A proximate to the main body portion 130 and further extends circumferentially about the entirety of the lens carrier 120 at the proximal end. In some embodiments, the lip does not extend the entire circumference of the main body portion. Rather, a smaller lip 150 or plurality of lips 150 extend inwardly toward the center axis A. A lip may be located at any convenient location at the proximal end of the lens carrier.

In some embodiments including ones where the lip extends circumferentially about the entirety of the lens carrier, the lip forms an aperture through which light passes into and back out from the lens. The aperture formed by the lip may be substantially circular. The aperture may be sufficiently large such that, if a procedure is being performed, the clinician will be able to adequately see through a sufficient portion of the lens to view relevant features in the patient's eye. In some embodiments, the lip forms an aperture through which a surgeon (e.g., using surgical equipment such as a scope or camera) views the eye through the lens.

The lip 150 has a lip lower surface 151, below which a hollow interior region 190 is formed by the sidewall 131 of the lens carrier 120. When the lens 110 is received by the lens carrier 120, a vertical travel of the lens 110 may be inhibited by the lip 150. In such a manner, a vertical position of the lens 110 in the lens carrier 120 is constrained by the lens upper surface 112 abutting the lip lower surface 151. Such constraint may be further effected by other features and embodiments described herein (snug fit of the lens and the lens carrier, rib(s)/ridge(s), rough surface(s), and/or the like).

Referring to FIG. 7, in some embodiments, the lip may have a radial length L₂ of between 2 and 20 mm, such as about 1 or 2 mm or about 1 to 3 mm. The lip 150 may have a height H₃ of between 0.5 and 2 mm in some embodiments. The diameter D₄ of the aperture of the lip may be between 8 and 12 mm or between about 7 and 11 mm, in some embodiments.

In some embodiments, the lens carrier may further comprise one or more tabs. The tab 160 is proximate to the first or proximal end of the lens carrier and extends outwardly from a center axis A of the ophthalmic device or the main body portion. In some embodiments, the tab extends away from the center axis of the ophthalmic device or main body portion. In some embodiments, the tab extends in a radial direction from the center axis.

For example, the tab 160 may be configured to allow a user to grip, grab, pinch, or otherwise engage the tab 160. The clinician may engage the tab 160 with fingers (e.g., thumb and forefinger) or with a surgical instrument or tool. Pulling or pushing on the tab allows the clinician to move the ophthalmic device thereby causing a corresponding movement of the ophthalmic device or the patient's eye. The tab is configured to facilitate movement of the eye by the clinician, for example, to perform aspects of a vitrectomy procedure. In this way, the lens carrier is configured to facilitate movement of the ophthalmic device at its first or proximal end.

In some embodiments, the tab may extend from a lip and/or from the main body sidewall. The tab may extend substantially horizontally from the center axis A of the ophthalmic device or may extend at an angle therefrom. The tab may be any suitable size or shape. For example, the tab may be substantially flat or may have a curvature or segments at different angles. Referring to FIG. 7, the tab may have a length L₃ of between about 2 and 20 mm, such as at least about 2, 3, 4, 5, 6, 7, or 8 mm.

The tab may have a texture on at least a portion of or all of its outer surface. Such a texture may facilitate the clinician engaging with the tab. The tab may have one or more features to facilitate the clinician engaging with the tab such as a recess for a finger or tool.

In some embodiments, the lens carrier may comprise a skirt (e.g., 170), also referred to as the skirt portion of the lens carrier. The skirt is proximate to and extends from the second or distal end of the lens carrier (e.g., near the distal end of the lens). The skirt is configured to contact the eye and facilitate movement of the ophthalmic device on the eye and/or to hold the eye during a procedure.

The skirt has a skirt lower surface (e.g., 171) that is configured to contact a surface of the patient's eye. The skirt can extend radially from the main body and/or the center axis. The skirt lower surface may have a contour that matches that of a portion of the cornea of the patient's eye, such as a human eye. The skirt lower surface may extend along the same direction as the lens lower surface. The skirt lower surface (and optionally the lens lower surface) may contact the patient's eye (or a fluid interposed between the eye and the skirt). Referring to FIG. 7, the radial length L₁ of the skirt may be between about 1 to 4 mm.

The skirt (and optionally the lens lower surface) can removably attach to the eye via a suction force. Because the skirt portion of the lens carrier is flexible, it facilitates a relatively light suction but also allows the lens to be repositioned with respect to the patient's eye with a relatively small force. With suction facilitated by the skirt portion, movement of the ophthalmic device may facilitate a corresponding movement of the eye. Thus, if the lens carrier (or the entire ophthalmic device) is moved, then the eye may move in a corresponding direction.

In some embodiments, the lip may assist in movement of the ophthalmic device. As shown, for example in FIG. 6, the lip may extend from the main body portion at the proximal end of the lens carrier. The lip may extend outwardly from the center axis A of the main body portion. In some embodiments, the lip extends circumferentially about the entirety of the lens carrier at the proximal end. In other embodiments, the lip does not extend the entire circumference of the main body portion. A smaller lip (or plurality of lips) extend toward the center axis A. A lip may be located at any convenient location at the proximal end of the lens carrier.

The lens carrier may be formed of or include any suitable material such as silicone, stainless steel, or various plastics and may be a molded lens carrier formed, for example, by injection or compression molding. Preferably, the material of the lens carrier is flexible and elastic and comprised of a material such as silicone or any other flexible and elastic material suitable for medical use, enhancing flexibility and maneuverability of the lens carrier. The lens carrier may be integrally formed (e.g., molded) as a single piece or portions of the lens carrier (e.g., main body 130, lip 150, tab 160, and/or skirt 170) or may be separately joined and integrated into a single lens carrier.

The lens and the lens carrier may be pre-assembled as a single assembly, or the clinician may insert the lens into the lens carrier. Whether the manufacturer or the clinician, the lens may be fitted into the lens carrier. The lens carrier may be sufficiently flexible (e.g., silicone) to allow the lens to be relatively easily loaded into the lens carrier. For example, the lens upper surface may be installed into the hollow interior region of the main body portion through the lower aperture or upper aperture in the main body portion.

In embodiments, particularly those employing flexible and elastic materials such as silicone, the lens is held snugly by the lens carrier. For example, the diameter(s) of the lens can be larger than the corresponding diameter(s) of the hollow interior region of the lens carrier, and the lens carrier would be stretched to accommodate the lens. For example, referring to FIG. 7, an inner diameter of the lens carrier (e.g., D₃) may be less an outer diameter of a lens (D₂) when the lens and lens carrier are unassembled. Alternatively, or in addition, the diameter of the aperture (e.g., D₄) may be less than the largest diameter of the lens (e.g., D2) or another diameter of the lens (e.g., D₁) when the lens and lens carrier are unassembled. In other embodiments, the diameter of the hollow interior region of the main body (D₃) may be larger than the diameter D₂ of the lens and/or the diameter of the hollow interior region D₄ may be larger than the largest diameter of the lens (e.g., D2) or another lens diameter (e.g., D₁).

It will be understood by those skilled in the art that various changes may be made and equivalents may be substituted without departing from the scope of the novel techniques disclosed in this application. In addition, many modifications may be made to adapt a particular situation or material to the teachings of the novel techniques without departing from its scope. Therefore, it is intended that the novel techniques not be limited to the particular techniques disclosed, but that they will include all techniques falling within the scope of the appended claims.

1. An ophthalmic device, comprising:
a contact lens including a lens upper surface and a lens lower surface, wherein the lens lower surface comprises a first curvature configured to contact an eye; and
a lens carrier configured to hold the contact lens, the lens carrier having a first end and a second end, and further comprising:
a main body portion comprising a sidewall configured to extend circumferentially about at least a portion of the contact lens, and a hollow interior region within the sidewall, wherein the main body portion is configured to receive at least a portion of the lens within the hollow interior region;
a tab proximate to the first end of the lens carrier and extending outwardly from a center axis of the main body portion; and
a skirt proximate to and extending from the second end of the lens carrier, wherein the skirt is configured to contact the eye.

2. The ophthalmic device of claim 1, wherein the contact lens is a vitrectomy lens.

3. The ophthalmic device of claim 1, wherein the lens upper surface includes a planar curvature.

4. The ophthalmic device of claim 1, wherein the lens carrier further comprises a lip extending above the hollow interior region at the first end, the lip extending inwardly towards the center axis of the main body portion.

5. The ophthalmic device of claim 4, wherein the lip forms an aperture to view the eye through the contact lens.

6. The ophthalmic device of claim 1, wherein the lens carrier is formed of a flexible material and is configured to flexibly fit the lens.

7. The ophthalmic device of claim 1, wherein the lens carrier is a molded lens carrier integrally formed as a single piece.

8. The ophthalmic device of claim 1, wherein an inner diameter of the lens carrier is less than an outer diameter of the lens.

9. The ophthalmic device of claim 1, wherein the tab extends in a radial direction from the center axis of the main body portion.

10. The ophthalmic device of claim 1, wherein a length of the tab is at least about 2 mm.

11. The ophthalmic device of claim 1, wherein the tab is configured to facilitate movement of the eye.

12. The ophthalmic device of claim 1, wherein the lens carrier further comprises a recess or rib on a sidewall of the main body portion and the lens further comprises a corresponding rib or recess on a lateral surface of the lens that engages the recess or rib of the lens carrier.

13. An ophthalmic device, comprising:
a vitrectomy lens including a lens upper surface and a lens lower surface, wherein the lens lower surface comprises a curvature configured to contact an eye; and
a lens carrier, comprising:
a main body portion including a sidewall and a hollow interior region, wherein the lens carrier receives at least a portion of the vitrectomy lens;
a lip at the proximal end of the lens carrier, the lip extending inwardly towards a center axis of the ophthalmic device.

14. The ophthalmic device of claim 13, wherein the lens carrier further comprises a tab extending away from the center axis of the ophthalmic device.

15. The ophthalmic device of claim 13, wherein the vitrectomy lens carrier further comprises a skirt portion configured to contact the eye.

16. The ophthalmic device of claim 15, wherein a length of the lip is between about 1 and 2 mm.

17. The ophthalmic device of claim 13, wherein the lip forms an aperture to view the eye through the vitrectomy lens.

18. The ophthalmic device of claim 13, wherein the lens carrier comprises a flexible material and is flexible.

19. The ophthalmic device of claim 13, wherein the lens carrier is a molded lens carrier integrally formed as a single piece.

20. The ophthalmic device of claim 19, wherein an inner diameter of the lens carrier is less than an outer diameter of the lens.

21. An ophthalmic device, comprising:
a lens carrier having a first end and a second end, wherein the lens carrier is configured to surround and hold at least a portion of a vitrectomy lens within an interior portion of the lens carrier,
the first end of the lens carrier being configured to facilitate movement of the device; and
the second end of the lens carrier being configured to facilitate contact between the vitrectomy lens and the eye; and
the lens carrier being further configured to retain the vitrectomy lens within the lens carrier when at least a portion of the ophthalmic device is manipulated.

22. The ophthalmic device of claim 21, wherein the lens carrier comprises a tab extending outwardly from a center axis of the ophthalmic device.

23. The ophthalmic device of claim 21, wherein the lens carrier comprises a main body portion and a lip, wherein:
the main body portion is configured to encompass a proximal end of the vitrectomy lens, and
the lip is located proximally from the main body portion, the lip being positioned proximally from the proximal end of the vitrectomy lens when the lens is within the carrier.

24. The ophthalmic device of claim 21, wherein the lens carrier is flexible and comprises silicone and wherein an inner diameter of the lens carrier is less than an outer diameter of the lens.
